(19) United States
(12) Patent Application Publication
(10) Pub. No.: US 2011/0282386 A1
(43) Pub. Date: Nov. 17, 2011

(54) CONTINUOUS-FILAMENT THREAD HAVING A PLURALITY OF BARBS AND A BARBED SUTURE

(75) Inventors: Volker Friedrich, Barcelona (ES); Erich Odermatt, Schaffhausen (CH); Lutz Funk, Sant Cugat der Valles (ES); Marta Casanovas Albalate, Barcelona (ES)

(73) Assignee: AESCULAP AG, Tuttlingen/Donau (DE)

(21) Appl. No.: 13/104,085

(22) Filed: May 10, 2011

(30) Foreign Application Priority Data
May 11, 2010 (EP) 10162574.7

(51) Int. Cl. A61B 17/04

(52) U.S. Cl. 606/228

(57) ABSTRACT

A continuous-filament thread includes a continuous-filament thread body including a plurality of barbs projecting from the thread body.

### RELATED APPLICATION

This application claims priority of European Patent Application No. 10162574.7, filed May 11, 2010, herein incorporated by reference.

### TECHNICAL FIELD

This disclosure relates to a continuous-filament thread having a plurality of barbs, a method for producing a continuous-filament thread having a plurality of barbs, a barbed suture and a method for producing a barbed suture.

### BACKGROUND

Various surgical methods employing sutures have been used in the past for closing or binding together wounds in human or animal tissue. More specifically, the surgeon may use a surgical needle with an attached conventional suture to pierce the tissue alternately on opposing faces of the wound and thus sew the wound closed. Usually, the sutures are knotted to obtain a secure “fix” in the tissue. Care has to be taken to ensure that the wounds to be closed are sutured with an optimal force at the wound margins. If the wound margins are sutured too loosely and too irregularly, for example, there is in principle a risk of increased scarring or dehiscence. In contrast, if the wound margins are sewed too strongly, there is a danger of the circulation of blood in the wound margins being restricted, which can result in necrotic changes in the surrounding tissue area.

In addition to the risk of possible complications, in particular further surgical interventions, there is therefore always a degree of risk of the wound repair, based on knotting of sutures, leading to impaired healing and to unsatisfactory cosmetic results in the patients concerned. Another consideration is that several knots often have to overlap to achieve a secure knot hold. This entails introducing a large amount of material into the area of the wound, which may elicit tissue erosion and foreign-body reactions.

Sutures which, in contrast to conventional sutures, do not have to be knotted have long been known as “barbed sutures.” Since the time of their conception, barbed sutures have offered numerous advantages over closing wounds with conventional sutures. A barbed suture typically includes an elongated body that has spaced barbs which project from the body surface along the body length. The barbs are arranged on the elongate body such that the suture can be pulled through the tissue along the direction of the barbs without any great resistance and without tissue trauma. When a pull is exerted in the opposite direction, however, the barbs stand upright and anchor themselves, and therefore also the suture, in the surrounding tissue area. This ensures that the suture cannot be pulled back through the incision channel. Thus, the main advantage of barbed sutures has been the provision of a non-slip attribute. Barbed sutures are described, for example, in U.S. Pat. No. 3,123,077 A, EP 1 559 266 B1, EP 1 560 683 B1 and EP 1 555 946 B1.

With respect to barbed sutures, the barbs are often arranged in a symmetrical fashion on the suture. For instance, the barbs may be formed in a helical pattern on the sutures, wherein the helix has a defined twist direction, cut depth and cut distance. However, such a helical disposition of the barbs may lead to a coil of the suture depending on the length of the incision, which may have an adverse effect on scarring.

A further disadvantage associated with barbed sutures relates to their production process. Normally, barbed sutures having distinct properties, in particular in view of barb geometry, have to be produced in an elaborate and time-consuming process.

It could therefore be helpful to provide a barbed material including a barbed suture which avoids known disadvantages. In particular, the barbed material should allow for a comfortable provision of barbed sutures having distinct properties depending on the wound to be closed by the barbed suture.

### SUMMARY

We provide a continuous-filament thread including a continuous-filament thread body including a plurality of barbs projecting from the thread body.

We also provide a barbed suture including at least one elongate suture body including a plurality of barbs which project from the at least one suture body and are formed from the continuous-filament thread.

We further provide a surgical instrument including a surgical needle and a surgical suture combination including the barbed suture.

### BRIEF DESCRIPTION OF THE DRAWINGS

FIG. 1 shows an example of a continuous-filament thread.

FIG. 2 shows a further example of a continuous-filament thread.

FIG. 3a shows an example of a barbed suture and surgical needle and barbed suture combination, respectively.

FIG. 3b shows a further example of a barbed suture and surgical needle and barbed suture combination, respectively.

FIG. 3c shows a further example of a barbed suture and surgical needle and barbed suture combination, respectively.

FIG. 3d shows a further example of a barbed suture and surgical needle and barbed suture combination, respectively.

FIG. 4 shows a further example of a barbed suture and surgical needle and barbed suture combination, respectively.

FIG. 5 shows a further example of a barbed suture and surgical needle and barbed suture combination, respectively.

FIG. 6 shows a field of application for an example of the surgical needle and barbed suture combination.

### DETAILED DESCRIPTION

We provide a continuous-filament thread comprising a continuous-filament thread body comprising barbs, in particular a plurality of barbs. The barbs preferably project from the continuous-filament thread body.

The terms “continuous-filament thread” and “continuous-filament thread body,” respectively, as used herein, preferably relates to a thread and thread body, respectively, that is unprocessed at least in view of its length (“endless-filament thread” and “endless-filament thread body,” respectively).

The term “barbs” as used herein encompasses at least one barb.

The term “plurality of barbs” as used herein encompasses at least two or more barbs.

The terms “thread” and “thread body,” respectively, as used herein may include a fiber, a yarn, a strand, a monofilament, in particular a pseudo-monofilament, or a multifilament, in particular a braided or intertwined multifilament.

The continuous-filament thread body may comprise a uniform barb configuration or a different or varying, in particular an alternating or a random barb configuration. The term “configuration” is to be understood in its broadest sense and in particular encompasses the design, shape, geometry and/or disposition of the barbs. More specifically, the barbs may have a uniform or different or varying, in particular alternating or random, configuration in view of barb length, barb height, cutting depth, cutting distance, cutting angle, cutting angle offset and combinations thereof. In view of producing barbed sutures having different or varying, in particular alternating or random, barb configurations, it is preferable that the continuous-filament thread body comprises a varying, in particular alternating or random, barb configuration along its length. Thus, a multiplicity of barbed sutures having distinct barb configurations may be produced from a single continuous-filament thread.

It is especially preferred that continuous-filament thread body comprises a random barb configuration.

Preferably, the barbs have a shape that is selected from the group consisting of escutcheon-shape, shield-shape, scale-shape, wedge-shape, thorn-shape, W-shape, arrow-shape, spike-shape, tin-shape, V-shape and, combinations thereof. Further, the barbs are preferably pointed or tapered at their free ends. Furthermore, the barbs may have a multi-tip configuration, in particular a twin-tip configuration. An example for barbs having a twin-tip configuration is the above mentioned W-shaped formation of barbs. Barbs having a twin-tip configuration may in particular be based on flat cuts into the continuous-filament thread body, preferably formed with a small angular offset and in small intervals from each other.

The barbs may be arranged in a uniform disposition on the continuous-filament thread body.

However, in view of the production of different barbed sutures from a single continuous-filament thread, it may be preferred that the barbs are arranged in different or varying, in particular alternating or, more preferable, random, dispositions on the continuous-filament thread body.

More specifically, the barbs may have a disposition on the continuous-filament thread body that is selected from the group consisting of a row disposition, a staggered disposition, an overlapping disposition, an offset disposition, an offset and partially overlapping disposition, a zigzag disposition, a random or arbitrary disposition, a meander-like disposition, a serpentine-like disposition, a sinus-like disposition, a spiral disposition, a helical disposition, and combinations thereof.

The barbs may be arranged in the form of left-handed and/or right-handed spirals and/or helices on the continuous-filament thread body. More specifically, left-handed and/or right-handed spirals and/or helices may be present on the continuous-filament thread body in a different or varying, in particular alternating or random, disposition. For example, a spiral or helical barb disposition having a different or varying, in particular alternating or random, spiral or helical twist, contributes to the reduction of suture coiling. Thus, adverse interactions between a tissue and a coiled suture may be significantly reduced, thereby enhancing wound healing.

Preferably, the barbs are arranged in a unidirectional disposition on the continuous-filament thread body. The term “unidirectional disposition” as used herein relates to a barb disposition, wherein the barbs are oriented in the same direction on the continuous-filament thread body.

More preferably, the barbs are arranged in a multidirectional, in particular bidirectional, disposition on the continuous-filament thread body. The term “multidirectional disposition” relates to a disposition of barbs, in which the barbs are oriented or aligned in multiple directions on the continuous-filament thread body. Accordingly, the term “bidirectional disposition” relates to a disposition of barbs in which the barbs are oriented or aligned in two different directions. Preferably, the barbs are oriented or aligned in opposing directions. More preferably, seen in the longitudinal direction of the continuous-filament thread body, the barbs for a first continuous-filament thread body section are preferably formed in the direction of a second continuous-filament thread body section and, for the second continuous-filament thread body section, are formed in the direction of the first continuous-filament thread body section.

The continuous-filament thread body may comprise repeating units of unidirectionally arranged barbs.

As an alternative or in combination, the continuous-filament thread body may comprise repeating units of multidirectionally arranged barbs. Preferably, the continuous-filament thread body comprises repeating units of bidirectionally arranged barbs. In particular, each unit may comprise a first section and a second section, wherein, seen in the longitudinal direction of the continuous-filament thread body, the barbs of the first section are oriented in the direction of the second section and the barbs of the second section are oriented in the direction of the first section.

Generally, the continuous-filament thread body may comprise at least two, in particular two or three, multidirectional dispositions of barbs.

The continuous-filament thread body may comprise at least two, in particular two, bidirectional dispositions of barbs on its surface. It is particularly preferable if, in relation to a first bidirectional disposition of barbs, a second bidirectional disposition of barbs is formed on the continuous-filament thread body at approximately 180° in the circumferential direction and preferably offset in relation to the first bidirectional disposition.

It is also possible for the continuous-filament thread body to comprise three bidirectional dispositions of barbs. In this case, it is preferable if, in relation to a first bidirectional disposition of barbs, a second bidirectional disposition of barbs is formed on the continuous-filament thread body at approximately 120° in the circumferential direction and preferably offset in relation to the first bidirectional disposition, which second bidirectional disposition of barbs is in turn formed at approximately 120° in the circumferential direction and preferably offset in relation to a third bidirectional disposition of barbs, such that the third bidirectional disposition of barbs is likewise formed at approximately 120° in the circumferential direction and preferably offset in relation to the first bidirectional disposition of barbs.

The continuous-filament thread body may comprise more than three, in particular four, five or six, multidirectional, in particular bidirectional, dispositions of barbs on the continuous-filament thread body.

Preferably, the continuous-filament thread body comprises sections that are free of barbs, in the following also denoted as barbless sections. The barbless sections may generally serve as potentially cutting areas to obtain barbed sutures from the continuous-filament thread body. Preferably, the barbless sections at least are also present in a barbed suture that is obtained from the continuous-filament thread. More specifically, the barbless sections may serve as junction areas for joining lengths or sections, in particular sutures bodies, obtained by cutting the continuous-filament thread body. Furthermore the barbless sections may serve as attachment areas for attaching medical instruments, in particular inserting instruments such as a surgical needle, and/or for attaching medical devices such as meshes to a barbed suture that may be obtained from the continuous filament thread. Furthermore, the barbless sections may advantageously facilitate the identification of the center of a barbed suture that may be obtained from the continuous-filament thread.

Terminal ends of the continuous-filament thread body may be free of barbs. More specifically, the continuous-filament thread body may comprise a different or varying, in particular an alternating or a random, disposition of barbed sections and barbless sections.

It is especially preferred that the continuous-filament thread body comprises sections having barbs that are interspaced by sections having no barbs. In other words, it is especially preferred that the continuous-filament thread body may comprise an alternating disposition of sections having barbs (barbed sections) and sections having no barbs (barbless sections). The barbed sections may comprise unidirectionally or multidirectionally, in particular bidirectionally, arranged barbs.

Preferably, the continuous-filament thread body is designed as a mass or solid continuous-filament thread body. This in particular means that the continuous-filament thread body is free of a lumen or the like. As already mentioned before, the continuous-filament thread body may be designed in the form of a fiber, yarn, strand, monofilament, pseudo-monofilament or multifilament, in particular braided or intertwined multifilament.

Alternatively, the continuous-filament thread body may be designed as a hollow continuous-filament thread body, in particular as a tubular continuous-filament thread body, preferably as continuous-tube or continuous-hose. The term “continuous-tube” and “continuous-hose,” respectively, preferably relates to a tube and hose, respectively, that is unprocessed at least in view of its length. Preferably, the hollow continuous-filament thread body comprises a closed wall, wherein the continuous-filament thread body preferably comprises open ends. A hollow, in particular tubular, continuous-filament thread body may be produced, by way of example, by an extrusion process. The barbs may project into the interior and/or exterior of the hollow continuous-filament thread body. Particularly, the barbs may be designed as cuts into the hollow continuous-filament thread body, wherein the barbs preferably do not break through the wall of the hollow continuous-filament thread body. As an alternative or in combination, the barbs may be designed as breakthroughs, i.e., the barbs are formed completely breaking through the wall of the hollow continuous-filament thread body.

Further, the continuous-filament thread body may not be limited in view of its cross section. In general, a circular cross section of the continuous-filament thread body is preferred. However, other cross-sectional shapes are likewise conceivable. For example, the continuous-filament thread body can have an oval, triangular, rosette-like, square, trapezoidal, rhomboid, pentagonal, hexagonal, star-shaped or cross-shaped cross section. Such cross-sectional shapes can readily be formed by suitable extrusions dies, which can be produced specific to a customer with any desired cross-sectional shape.

The thread body may comprise at least one, in particular one, set of barbs. The barbs may also be configured in at least two, in particular two, three, four, five or more, sets, each set having a barb configuration different from the barb configuration of the other set.

Preferably, the continuous-filament thread body is made from a material selected from the group consisting of a bio-absorbable material, a non- absorbable material, and combinations thereof. Typically, the continuous-filament thread body is made of a polymer. The term “polymer” as used herein may include copolymers, in particular random copolymers, alternating copolymers, graft copolymers and/or block copolymers. Further, the term “copolymer” relates to a polymer that is composed of at least two different monomer units. Accordingly, the continuous-filament thread body may also be made from terpolymers, tetrapolymers or the like.

Preferably, the continuous-filament thread body is made of a polyhydroxyalkanoate, a copolymer thereof or a mixture thereof. More specifically, the continuous-filament thread body is preferably made of a bio-absorbable material that is selected from the group consisting of polylactide, polyglycolide, polydioxanone, polytrimethylene carbonate, poly-ε-caprolactone, poly-3-hydroxybutyrate, poly-4-hydroxybutyrate, copolymers thereof and mixtures thereof.

The continuous-filament thread body may be made of a non-absorbable material that is selected from the group consisting of polyamide, polyester such as polyethylene terephthalate, polypropylene terephthalate and/or polybutylene terephthalate, polyethylene, polypropylene, polyurethane, polytetrafluoroethylene, in particular expanded polytetrafluorethylene, polyvinylidene difluoride, polytetrafluoropropylene, polyhexafluoropropylene, polyetherester, copolymers thereof, and mixtures thereof.

Furthermore, the continuous-filament thread body may comprise additives such as biological agents, medical agents, pharmaceutical agents, cells, and combinations thereof. Biological active agents may be selected from the group consisting of differentiation factors, growth factors, recruiting factors, adhesion factors, and combinations thereof. Appropriate growth factors may be selected from the group consisting of fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insuline-like growth factor (IGF), hepatocyte growth factor (HGF), interleucin-1 B (IL-1 B), interleucin-8 (IL-8), nerve growth factor (NGF), and combinations thereof. Medical and pharmaceutical agents, respectively, may be selected from the group consisting of antimicrobial agents, in particular antibiotic agents, disinfecting agents, growth-promoting agents, anti-inflammatory agents, analgetic agents, odor-controlling agents, and mixtures thereof. Preferred cellular additives may be somatic cells, in particular autologous cells, like fibroblasts, chondrocytes and/or precursors cells, in particular stem cells, for example. Furthermore, such substances, like collagen and/or hyaluronic acid, for example, may be particularly beneficial in the field of plastic surgery to achieve cosmetically satisfactory results, like a smoothing of wrinkles, for example.

The continuous-filament thread body may be present in a drawn or undrawn state. The continuous-filament thread body may also be present in a sterile form.

We also provide a method for producing a continuous-filament thread comprising a continuous-filament thread body comprising barbs, in particular a plurality of barbs, which preferably project from the continuous-filament thread body, wherein the barbs are cut into a barbless continuous-filament thread body, i.e., the barbs are cut into a continuous-filament thread body having no barbs.

The barbs may be formed using any suitable method, including cutting, in particular mechanical cutting, injection molding, stamping, laser, and the like.

Further, the barbs may be cut into an undrawn or into a drawn continuous-filament thread body.

Preferably, the barbs are cut in while the continuous-filament thread body is rotated. The continuous-filament thread body is twisted prior to cutting in barbs and untwisted again after the barbs have been cut in. The structures described in this paragraph are advantageous in that a radial, in particular spiral-shaped or helical-shaped disposition of barbs may be generated on the continuous-filament thread body.

For cutting, in particular mechanical cutting, conventional cutting devices comprising a cutting bed, at least one cutting plate and holding or fixing elements such as vices, chucks, holding or clamping jaws, may be used. For mechanical cutting of the barbs, it is particularly preferable to use a cutting bed with a groove, wherein the groove is provided to receive the continuous-filament thread body that is to be cut. Depending on the depth of the groove, it is possible, when using at least one cutting plate, to specifically influence the cutting depth with which the barbs are cut into the continuous-filament thread body. This is because the at least one cutting plate is generally designed such that, by means thereof, cuts can be made only in the areas of the continuous-filament thread body, which protrude from the groove. This advantageously contributes to a safe cutting.

Lasers that can be used are, in principle, gas lasers, for example, CO₂ lasers, and also solid-state lasers, for example, Nd:YAG lasers (neodymium-doped yttrium aluminium garnet lasers). A suitable laser cutting machine generally comprises a laser beam source, a beam guide, and at least movable focusing lens (concave mirror or positive lens). The beam leaving the beam source is either guided by fiber-optic cables, for example, in a Nd:YAG laser, or by a deflecting mirror, for example, in a CO₂ laser, to the machining lens which focuses the laser beam and in this way generates the power densities needed for the cutting, generally in the range of 10⁶ to 10⁹ W/cm². Corresponding cutting laser methods are sufficiently known such that further details are not given here.

With respect to a drawing of the continuous-filament thread body, such a drawing may be performed in a temperature range of between 20° and 80° C. above the glass transition temperature of the thread body material. For instance, infrared radiation can be used, to generate a suitable heat for the drawing process. To draw the continuous-filament thread body, the thread body is usually guided across a roller system, so-called “drawing frame,” in which the rollers can have different speeds of rotation. Generally, each subsequent roller has a higher speed of rotation than the proceeding roller of the drawing system. As an alternative to the continuous drawing just described, it is also possible to carry out intermittent drawing. For intermittent drawing, the thread body may be clamped between the clamping jaws of a tensioning device and then drawn. For drawing the continuous-filament thread body, a drawing ratio of between 2.5 and 8 and particularly of between 3 and 5 may be chosen.

After the continuous-filament thread body has been drawn, the thread body can be subjected to various post-treatment steps. For this purpose, the continuous-filament thread body is generally tempered (heat-treated) in a vacuum or reduced pressure atmosphere. In this way, crystallinity of the continuous-filament thread body may be increased and the residual monomer content reduced. A further advantage afforded by a post-treatment of the continuous-filament thread body material is the reduced susceptibility to shrinkage.

We further provide to a barbed suture comprising at least one elongate suture body comprising barbs, in particular a plurality of barbs, which preferably project from the at least one suture body. The barbed suture is preferably obtainable or obtained from a continuous-filament thread.

The term “at least one suture body” includes one, two, three, four, five, six, seven eight or more suture bodies. Depending on the number of suture bodies, the barbed suture may, by way of example, be arranged as a linear barbed suture, as star-like barbed suture or as a branched, in particular dendrimer-like, barbed suture.

In principle, the at least one suture body may comprise a uniform barb configuration or a different or varying, in particular an alternating or, more preferable, a random, barb configuration. More specifically, the barbs may have a uniform or different or varying, in particular alternating or random, configuration in view of barb design, barb geometry and/or barb shape, in particular in view of barb length, barb height, cutting depth, cutting distance, cutting angle, cutting angle offset and, combinations thereof.

Preferably, the barbs have a shape that is selected from the group consisting of escutcheon-shape, shield-shape, scale-shape, wedge-shape, thorn-shape, W-shape, arrow-shape, spike-shape, tin-shape, V-shape and, combinations thereof. Further, the barbs are preferably pointed or tapered at their free ends. Furthermore, the barbs may have a multi-tip configuration, in particular a twin-tip configuration. An example for barbs having a twin-tip configuration is the above mentioned W-shaped formation of barbs. Barbs having a twin-tip configuration may in particular be based on flat cuts into the at least one suture body, preferably formed with a small angular offset and in small intervals from each other.

The barbs may be arranged in a uniform disposition on the at least one suture body. However, different or varying, in particular alternating or random, dispositions may also be within the scope of this disclosure.

More specifically, the barbs may have a disposition on the at least one suture body that is selected from the group consisting of a row disposition, a staggered disposition, an overlapping disposition, an offset disposition, an offset and partially overlapping disposition, a zigzag disposition, a random or arbitrary disposition, a meander-like disposition, a serpentine-like disposition, a sinus-like disposition, a spiral disposition, a helical disposition, and combinations thereof.

The barbs may be arranged in the form of left-handed and/or right-handed spirals and/or helices on the at least one suture body. More specifically, left-handed and/or right-handed spirals and/or helices may be present on the at least one suture body in a different or varying, in particular alternating or random, disposition. For example, a spiral or helical barb disposition having a different or varying, in particular alternating or random, spiral or helical twist, contributes to the reduction of suture coiling. Thus, adverse interactions between a tissue and a coiled suture may be significantly reduced, thereby enhancing wound healing.

Preferably, the barbs are arranged in a unidirectional disposition on the at least one suture body.

More preferably, the barbs are arranged in a multidirectional, in particular bidirectional, disposition on the at least one suture body. Preferably, the barbs are orientated or aligned in opposing directions. More preferably, seen in the longitudinal direction of the at least one suture body, the barbs for a first suture body section are preferably formed in the direction of a second suture body section and, for the second suture body section, are formed in the direction of the first suture body section.

The at least one suture body may comprise repeating units of unidirectionally arranged barbs.

As an alternative or in combination, the at least one suture body may comprise repeating units of multidirectionally arranged barbs. Preferably, the at least one suture body comprises repeating units of bidirectionally arranged barbs. In particular, each unit may comprise a first section and a second section, wherein, seen in the longitudinal direction of the at least one suture body, the barbs of the first section are orientated in the direction of the second section and the barbs of the second section are orientated in the direction of the first section.

Generally, the at least one suture body may comprise at least two, in particular two or three, multidirectional dispositions of barbs.

The at least one suture body may comprise at least two, in particular two, bidirectional dispositions of barbs on its surface. It is particularly preferable if, in relation to a first bidirectional disposition of barbs, a second bidirectional disposition of barbs is formed on the at least one suture body at approximately 180° in the circumferential direction and preferably offset in relation to the first bidirectional disposition.

It is also possible for the at least one suture body to comprise three bidirectional dispositions of barbs. In this case, it is preferable if, in relation to a first bidirectional disposition of barbs, a second bidirectional disposition of barbs is formed on the at least one suture body at approximately 120° in the circumferential direction and preferably offset in relation to the first bidirectional disposition, which second bidirectional disposition of barbs is in turn formed at approximately 120° in the circumferential direction and preferably offset in relation to a third bidirectional disposition of barbs, such that the third bidirectional disposition of barbs is likewise formed at approximately 120° in the circumferential direction and preferably offset in relation to the first bidirectional disposition of barbs.

The barbs may be in principle arranged user-defined on the at least one suture body. For instance, the barbs may be arranged in the center of the at least one suture body.

Preferably, the at least one suture body comprises sections that are free of barbs, in the following also denoted as barbless sections.

More specifically, the at least one suture body may comprise a barbless section around the center of the at least one suture body. The barbless section may expediently serve as a control or reference section, which facilitates the identification of the barbed suture's center. Further, the barbless section may serve as a control or reference section to avoid an accidental sliding of the at least one suture body beyond its center into a tissue to be treated.

Further, the at least one suture body may have at least one terminal section (terminal end), in particular one, two or, if appropriate, more than two terminal sections, that is free of barbs. These barbless sections may beneficially serve as attachment areas for attaching the at least one suture body to at least one further suture body. Furthermore, the barbless sections may serve as attachment areas for attaching medical instruments, in particular inserting instruments such as a surgical needle and/or for attaching medical devices such as meshes to the at least one suture body.

The at least one suture body may comprise a different or varying disposition of barbed sections and barbless sections. Preferably, the at least one suture body comprises sections having barbs that are interspaced by sections having no barbs. More specifically, the at least one suture body may comprise an alternating disposition of sections having barbs and sections having no barbs. The barbed sections may comprise unidirectionally or multidirectionally, preferably bidirectionally, arranged barbs.

More specifically, the at least one suture body may have a first barbless terminal section, which is followed by a barbed section, which followed by a barbless section, which is followed by a barbed section, which is followed by a second terminal section.

Preferably, the at least one suture body is designed as at least one mass or solid suture body. More specifically, the at least one suture body may be designed in the form of a fiber, yarn, strand, monofilament, pseudo-monofilament or multifilament, in particular braided or intertwined multifilament.

Alternatively, the at least one suture body may be designed as at least one hollow suture body, in particular as at least one tubular suture body. Preferably, the at least one hollow suture body comprises a closed wall. Preferably, the at least one hollow suture body comprises open ends. Hollow suture bodies may be produced, by way of example, by an extrusion process. The barbs may project into the interior and/or exterior of the at least one hollow suture body. Preferably, the barbs protrude into the exterior of the at least one hollow suture body. Particularly, the barbs may be designed as cuts into the at least one hollow suture body, wherein the barbs do not break through the wall of the at least one hollow suture body. As an alternative or in combination, the barbs may be designed as breakthroughs, i.e., the barbs are formed completely breaking through the wall of the at least one hollow suture body. Hollow suture bodies may be used as self-anchoring delivery tubes, drainage tubes, catheters, drug delivery systems or the like.

Further, the at least one suture body may not be limited in view of its cross section. In general, a circular cross section of the at least one suture body is preferred. However, other cross-sectional shapes are likewise conceivable. For example, the at least one suture body can have an oval, triangular, rosette-like, square, trapezoidal, rhomboid, pentagonal, hexagonal, star-shaped or cross-shaped cross section. Such cross-sectional shapes can readily be formed by suitable extrusions dies, which can be produced specific to a customer with any desired cross-sectional shape.

The at least one suture body may comprise at least one, in particular one, set of barbs. The barbs may also be configured in at least two, in particular two, three, four, five or more, sets, each set having a barb configuration different from the barb configuration of the other set.

Preferably, the at least one suture body is made from a material selected from the group consisting of a bio-absorbable material, a non-absorbable material, and combinations thereof. Typically, the at least one suture body is made of a polymer.

Preferably, the at least one suture body is made of a polyhydroxyalkanoate, a copolymer thereof or a mixture thereof. More specifically, the at least one suture body is preferable made of a bio-absorbable material that is selected from the group consisting of polylactide, polyglycolide, polydioxanone, polytrimethylene carbonate, poly-ε-caprolactone, poly-3-hydroxybutyrate, poly-4-hydroxybutyrate, copolymers thereof and mixtures thereof.

The at least one suture body may be made of a non-absorbable material that is selected from the group consisting of polyamide, polyester such as polyethylene terephthalate, polypropylene terephthalate and/or polybutylene terephthalate, polyethylene, polypropylene, polyurethane, polytetrafluoroethylene, in particular expanded polytetrafluorethylene, polyvinylidene difluoride, polytetrafluoropropylene, polyhexafluoropropylene, polyetherester, copolymers thereof, and mixtures thereof.

Furthermore, the at least one suture body may comprise additives such as biological agents, medical agents, pharmaceutical agents, cells, and combinations thereof. Biological active agents may be selected from the group consisting of differentiation factors, growth factors, recruiting factors, adhesion factors, and combinations thereof. Appropriate growth factors may be selected from the group consisting of fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insuline-like growth factor (IGF), hepatocyte growth factor (HGF), interleucin-1 B (IL-1 B), interleucin-8 (IL-8), nerve growth factor (NGF), and combinations thereof. Medical and pharmaceutical agents, respectively may be selected from the group consisting of antimicrobial agents, in particular antibiotic agents, disinfecting agents, growth-promoting agents, anti-inflammatory agents, analgetic agents, odor-controlling agents, and mixtures thereof. Preferred cellular additives may be somatic cells, in particular autologous cells, like fibroblasts, chondrocytes and/or precursors cells, in particular stem cells, for example. Furthermore, such substances, like collagen and/or hyaluronic acid, for example, may be particularly beneficial in the field of plastic surgery to achieve cosmetically satisfactory results, like a smoothing of wrinkles, for example.

The at least one suture body may be present in a drawn or undrawn state. The at least one suture body may be present in a sterile form.

At least one, in particular one, terminal section of the at least one suture body may be attached to a surgical needle.

Further, the suture, in particular the at least one suture body, may comprise two terminal sections, wherein both terminal sections are attached to one surgical needle, preferably forming a loop.

As an alternative, the suture, in particular the at least one suture body, may comprise two terminal sections, wherein each terminal section is attached to a surgical needle. Each terminal section may be attached to the same type of surgical needle or to a different type of surgical needle.

Instead of the surgical needle mentioned in the above-described structures, other surgical instruments, in particular introducing or inserting instruments, like sheaths, tubes or the like, may be attached to the suture, in particular the at least one suture body.

The suture, in particular the at least one suture body, may comprise at least one junction, in particular at least one chemical or physical, in particular mechanical, junction and/or at least one resistance element. The term “at least one junction” may include one, two or more than two junctions. The term “at least one resistance element” may include one, two or more than two resistance elements. The term “resistance element” preferably includes an element that contributes to an increased resistance, when the suture, in particular the at least one suture body, is pulled in and/or through a biological tissue.

More specifically, the suture, in particular the at least one suture body, may comprise a junction and/or resistance element that is present in the center or basically in the center of the suture, in particular the at least one suture body. Such a junction and/or resistance element advantageously serve as a control or reference point or element for the surgeon to be able to identify the center of the suture, in particular the at least one suture body. Further such a junction and/or resistance element expediently avoid an accidental sliding of the suture, in particular of the at least one suture body, beyond its center, when the suture is pulled in and/or through a biological tissue.

The at least one junction is preferably selected from the group consisting of a knot, a knob, a welded junction, in particular an ultrasonic welded junction, a soldered junction, a bonded junction, a joining flange, a T-fitting, and combinations thereof.

With respect to a chemical, in particular bonding junction, such a junction may be achieved using a molten polymer and/or an adhesive. Further, a bonding junction may be achieved by using bio-absorbable materials, non-absorbable materials, and/or combinations thereof.

Appendages of the junctions such as knot ears may be minimized, encapsulated or covered. For example, the appendages may be encapsulated or covered using a molten polymer and/or an adhesive. In particular, the appendages may be minimized to such an extent that they are no longer visible.

The at least one resistance element is preferably selected from the group consisting of a mold such as a plate, a disc, a spherical, a ring, a circular mold, a pad, a pledget, a textile such as a woven fabric, braided fabric, knitted fabric, a mesh or the like, a joining flange, a T-fitting, and combinations thereof. The at least one resistance element may be further present in a miniaturized form.

The barbs may be oriented or aligned towards the at least one junction and/or towards the at least one resistance element.

The at least one junction and/or the at least one resistance element may be made of a bio-absorbable material, non-absorbable material and/or combinations thereof. With respect to suitable materials, reference is made in its entirety to the previous description.

The barbed suture may comprise a plurality of suture bodies, wherein the suture bodies are preferably joined by at least one, in particular one, common junction and/or resistance element. The suture bodies may be made from a bio-absorbable material, a non absorbable material, and combinations thereof. Further, some of the suture bodies may be free of barbs.

Preferably, the barbed suture as already mentioned is designed as a linear barbed suture. More specifically, the barbed suture may comprise at least two, in particular two, suture bodies that are joined by at least one, in particular one, common junction and/or resistance element. Preferably, each suture body comprises an unidirectional or a multidirectional, in particular a bidirectional, disposition of barbs. More preferably, suture sections comprising an unidirectional or multidirectional, in particular bidirectional, disposition of barbs may be interspaced by barbless suture sections. More preferably, the suture comprises a barbless section around the common junction and/or resistance element. Further, it is preferred that the suture comprises terminal sections that are free of barbs. With respect to further details and advantages, in particular in view of the junction and/or resistance element, reference is made in its entirety to the previous description.

In case that the suture comprises more than two suture bodies, the barbed suture may be arranged in a non-linear, in particular in a star-like or branched, preferably dendrimer-like, fashion. In particular, the suture bodies may extend radially from a common junction and/or resistance element. With respect to further details and advantages, in particular in view of the junction and/or resistance element, reference is made in its entirety to the previous description.

The suture, in a particular the at least suture body, may comprise a marking, preferably in the form of a junction and/or resistance element, preferably for locating the center of the suture, in particular for locating the center of the at least one suture body. With respect to further details and advantages, in particular in view of the junction and/or resistance element, reference is made in its entirety to the previous description.

Further, the barbed suture, in particular the at least one suture body, may comprise a resistance element, in particular a stop element such as a knot, at one terminal section of the suture, in particular the at least one suture body. In particular, the suture, in particular the at least one suture body, may also comprise a mechanical junction such as a joining flange or a T-fitting at a terminal section of the suture, in particular the at least one suture body.

Preferably, the suture may comprise at least one mass or solid suture body. This in particular means that the at least one suture body is free of a lumen or the like. In particular, the at least one suture body may be designed as a fiber, monofilament, pseudomonofilament or multifilament, in particular as a braided or intertwined multifilament.

As an alternative, the suture may comprise at least one hollow suture body, in particular at least one tubular or hose-like suture body. The at least one hollow suture body preferably comprises a closed wall encasing a lumen. Further, the at least one hollow suture body may comprise open ends. The barbs may be designed as cuts into the at least one hollow suture body, wherein the cuts do not break through the wall of the at least one hollow suture body. As an alternative or in combination, the barbs may be designed as breakthroughs, i.e., are formed completely breaking through the wall of the at least one hollow suture body. Further, the barbs may protrude into the interior and/or exterior, preferably exterior, of the at least one hollow suture body. A barbed suture comprising at least one hollow, in particular at least one tubular or hose-like, suture body may be used as self-anchoring delivery tubes, drainage tubes, catheters, drug-release systems or the like.

The barbed suture may generally be used as a self-anchoring or knotless suture, which is due to the barbs that serve as anchoring structures towards biological, in particular human and/or animal, tissue. The barbed suture is in particular suitable for connecting and/or adaption of biological tissues. The tissue may include hard tissue like bone or cartilage as well as soft tissue like connective tissue. The tissues can be, for example, skin, fat, fascia, bones, muscles, organs, nerves, blood vessels, connective tissues, tendons or ligaments. The barbed suture may be in particular used in the field of endoscopic surgery, in particular laparoscopic surgery, microsurgery and/or plastic surgery. In the field of plastic surgery, the barbed suture may be employed for eye brow lifting and/or skin lining.

Furthermore, the barbed suture may be used for fixing medical devices such as medical meshes, in particular hernia meshes, prolaps meshes and/or urinary incontinence meshes. To support the fixation of medical devices, the barbed suture, in particular several barbed sutures, may be fixed to an fixation means like a ring.

We further provide a method for producing a barbed suture comprising at least one elongate suture body comprising barbs, in particular a plurality of barbs, which preferably project from the at least one suture body, wherein a continuous-filament thread comprising a continuous-filament thread body comprising barbs, in particular a plurality of barbs, which preferably project from the continuous-filament thread body, is cut into lengths or sections, in particular suture bodies.

Two or more of the cut lengths or sections, in particular suture bodies, are joined by a junction and/or resistance element, in particular a common junction and/or resistance element. With respect to further details and advantages of the method, reference is made in its entirety to the previous description.

We still further provide a surgical instrument, in particular surgical needle, and suture combination comprising a barbed suture. With respect to further details and advantages of the combination, in particular with respect to the barbed suture, reference is made in its entirety to the previous description.

Further features will become clear from the following description of the drawings and examples. The individual features can be realized either singly or severally in combination. The drawings and examples serve merely for illustration and better understanding and are not to be understood as in any way limiting this disclosure.

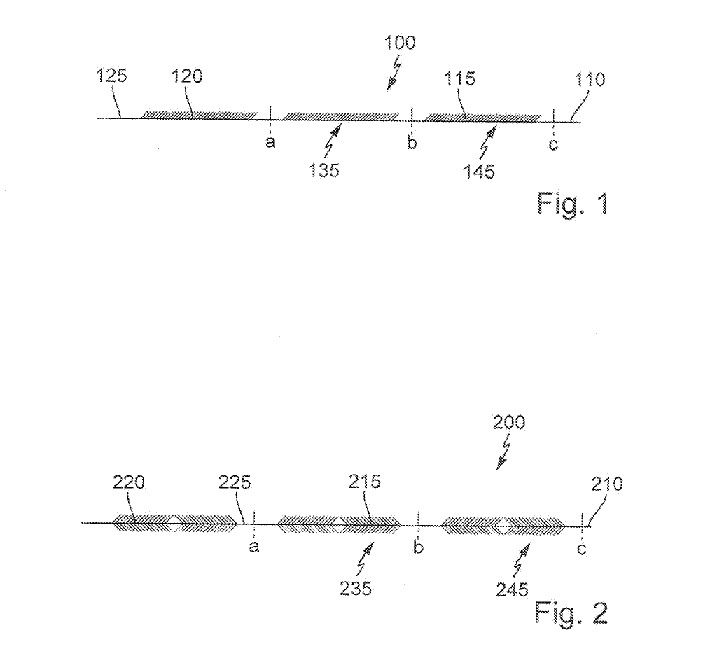

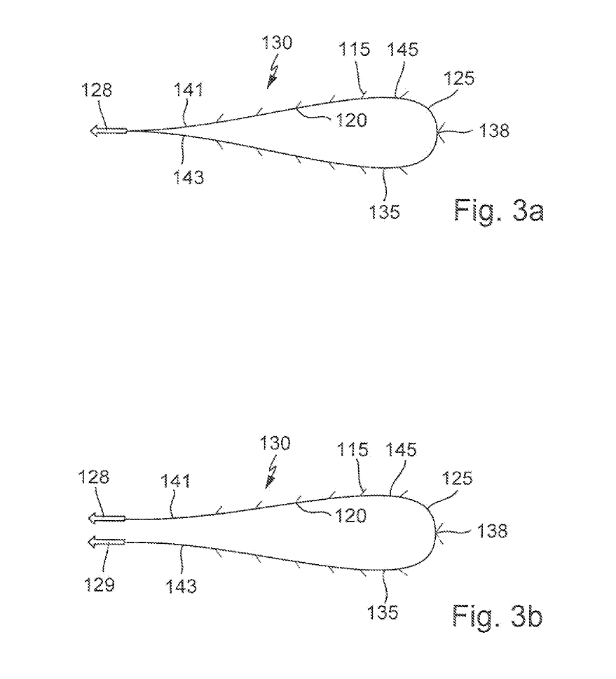

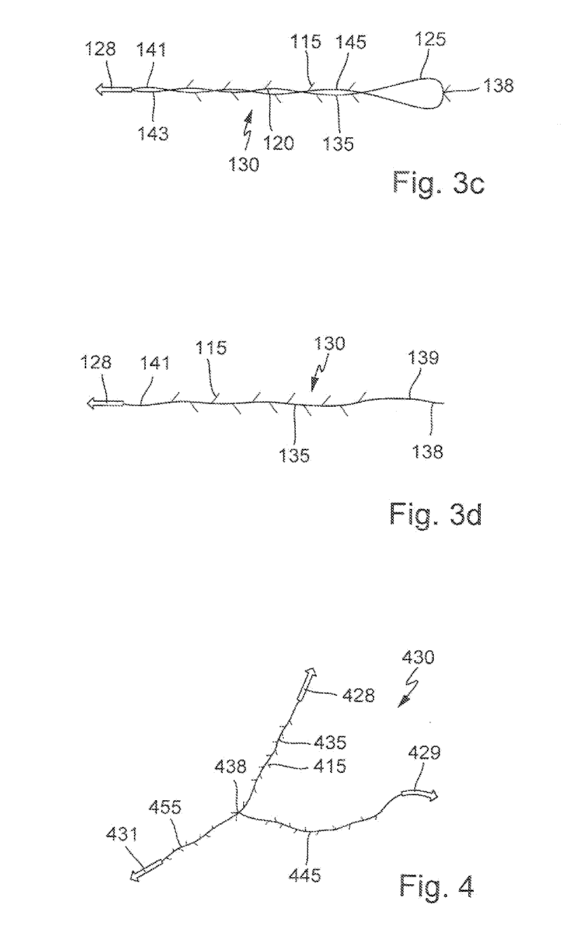

FIG. 1 discloses a continuous-filament thread 100 comprising a continuous-filament thread body 110. The continuous-filament thread body 110 comprises a plurality of barbs 115 projecting from the continuous-filament thread body 110. The barbs 115 are arranged in a unidirectional disposition on the continuous-filament thread body 110. More specifically, the continuous-filament thread body 110 comprises sections 120 having barbs hereinafter referred to as barbed sections and sections 125 having no barbs hereinafter referred to as barbless sections. The barbed sections 120 are interspaced by the barbless sections 125 and vice versa. The barbless sections 125 may advantageously serve as potential cutting areas for producing barbed sutures. For example, by cutting the continuous-filament thread body 110 at the dotted positions a-c, barbed sutures bodies 135, 145 may be produced as integral entities, i.e., in one piece, having unidirectionally arranged barbs. The suture bodies 135, 145 may be also joined in a subsequent step yielding a barbed suture 130 comprising bidirectionally arranged barbs as depicted in FIG. 3a.

FIG. 2 discloses a continuous-filament thread 200 comprising a continuous-filament thread body 210 comprising a plurality of barbs 215, which project from the continuous-filament thread body 210. The barbs 215 are arranged bidirectionally on the continuous-filament thread body 210. More specifically, the continuous-filament thread body 210 comprises barbed sections 220 having barbs and barbless sections 225, wherein the barbed sections 220 are interspaced by the barbless sections 225 and vice versa. By cutting the continuous-filament thread 210 at the dotted positions a-c, suture bodies 235, 245 having bidirectionally arranged barbs may be produced as integral entities, i.e., in one piece. The suture bodies 235, 245 may be joined in a subsequent step yielding a barbed suture comprising bidirectionally arranged barbs.

FIG. 3a shows a combination of a surgical needle 128 (depicted as an arrow) and a barbed suture 130 that comprises two suture bodies 135, 145 that are joined by a common junction 138 such as a knot. Both suture bodies 135, 145 comprise a plurality of unidirectionally arranged barbs 115, wherein the barbs 115 of the first suture body 135 are oriented in the direction of the second suture body 145 and the barbs 115 of the second suture body 145 are oriented in the direction of the first suture body 135. Thus, the barbed suture 130 in total comprises bidirectionally arranged barbs.

The barbed suture comprises barbed sections 120 and barbless sections 125. The barbless sections 125 are present around the common junction 138 and at the terminal sections 141, 143 of the suture 130. The terminal sections 141, 143 are attached to the surgical needle 128, preferably forming a suture loop as disclosed in FIG. 3.

The common junction 138 serves advantageously as a reference or control point for the surgeon to facilitate the identification of the suture's center. Furthermore, the common junction 138 may serve as a control element to avoid an accidental sliding of the barbed suture 130 beyond its center.

FIG. 3b discloses a further example of a surgical needle and a barbed suture combination. The terminal sections 141, 143 of the barbed suture 130 are attached to the surgical needles 128, 129, preferably forming a loop. The surgical needles 128,129 may be of the same type or may stand for different needle types. With respect to further details and advantages, reference is made in its entirety to the description belonging to FIG. 3a.

FIG. 3c discloses a further example of a surgical needle and a barbed suture combination. The barbed suture 130 comprises two suture bodies 135, 145 that are joined by a mutual junction 138. Both suture bodies 135, 145 comprise a section 120 of unidirectionally arranged barbs 115, wherein the barbed sections 120 are interspaced by a barbless section 125, wherein the barbless section 125 is around the mutual junction 138. Furthermore, the barbed suture 130 comprises terminal sections 141, 143 that are free of barbs and that are attached to the surgical needle 128. Besides, the suture bodies 135, 145 are twisted along the barbed sections 120. With respect to further details and advantages, reference is made in its entirety to the description belonging to FIG. 3a.

FIG. 3d discloses a further example of a surgical needle and barbed suture combination. The barbed suture 130 comprises a suture body 135 having a plurality of unidirectionally arranged barbs 115. The barbs 115 are present one the suture body 135 in form of two unidirectional dispositions. In relation to one barb disposition, the other disposition is formed on the suture body 135 at approximately 180° in the circumferential direction and offset in relation to the first unidirectional disposition. The barbed suture 130 comprises a junction 138 such as a knot at one terminal section 139 of the suture body 135. The opposite terminal section 141 is attached to a surgical needle 128. With respect to further details and advantages, reference is made in its entirety to the description belonging to FIG. 3a.

FIG. 4 shows a further example of a surgical needle and barbed suture combination. The barbed suture 430 comprises three suture bodies 435, 445, 455, wherein the suture bodies 435, 445, 455 comprise a plurality of barbs 415 and wherein the suture bodies 435, 445, 455 are joined by a mutual junction 438. The suture bodies 435, 445, 455 are roughly directed in the edges of a triangle. The mutual junction 438 may be a knot or as an alternative a T-fitting. The suture bodies 435, 445, 455 are attached to the surgical needles 428, 429, 431 as disclosed in FIG. 4. With respect to the surgical needles 428, 429, 431, needles of the same or a different type may be used. Furthermore, the suture bodies 435, 445, 455 may comprise barbed and/or unbarbed sections. Some of the suture bodies may be free of barbs 415. With respect to further features and advantages, reference is made in its entirety to the previous descriptions.

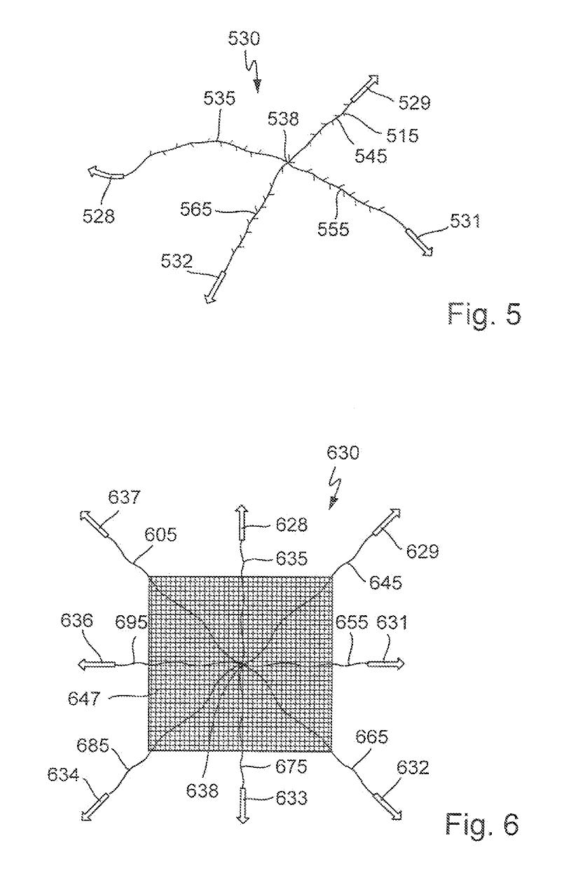

FIG. 5 shows a further example of a surgical needle and barbed suture combination. The barbed suture 530 comprises four suture bodies 535, 545, 555, 565, wherein the suture bodies 535, 545, 555, 565 comprise a plurality of barbs 515 and wherein the suture bodies 535, 545, 555 are joined by a mutual junction 538. The suture bodies 535, 545, 555, 565 are roughly directed in the edges of a square. The mutual junction 538 may be a knot. The suture bodies are attached to the surgical needles 528, 529, 531, 532. With respect to further features and advantages reference is made in its entirety to the previous descriptions.

FIG. 6 shows a star-like arranged barbed suture 630, wherein the barbs for sake of clearness are not depicted. The barbed suture comprises suture bodies 605, 635, 645, 655, 665, 675, 685, 695 being attached to the surgical needles 628, 629, 631, 632, 633, 634, 636, 637. The barbed suture 630 may be, by way of example, used to fix a medical mesh 647 in the body of the patient.

Instead of the junction described in the above descriptions, a resistance element may also be used.

### EXAMPLE

A polydioxanone monofilament is extruded, passed through a quench bath and wound on a drum winder without drawing the fiber to its final length. Subsequently the undrawn fiber is passed through a cutting device to produce an undrawn, continuous monofilament having a regular pattern of unidirectional barbed sections followed by sections that have no barbs. Afterwards this continuous barbed monofilament is drawn to its final length by passing it through multistage drawing equipment, applying a 3-fold drawing ratio and then wound on a spool. In a next step, the fiber is wound off the spool and cut in the middle of each unbarbed (barbless) section, resulting in an amount of monofilament strands having an unidirectional barbed section in the center and non-barbed sections at the ends of the strands.

These strands are then used to produce a star shaped fixation device for surgical meshes. Therefore four of these unidirectional strands as mentioned above are connected on one end by a knot, in a way that the directions of the barbs of the respective fibers points towards the knot. In a second step the other free fiber ends are attached to a needle resulting in the desired device (see FIG. 5).

1. A continuous-filament thread comprising a continuous-filament thread body comprising a plurality of barbs projecting from the thread body.

2. The continuous-filament thread according to claim 1, wherein the thread body comprises a uniform barb configuration or a random barb configuration, wherein the barbs have a shape selected from the group consisting of escutcheon-shape, shield-shape, scale-shape, wedge-shape, thorn-shape, W-shape, arrow-shape, spike-shape, tin-shape V-shape and, combinations thereof.

3. The continuous-filament thread according to claim 1, wherein the barbs are arranged in a uniform disposition or in a random disposition on the continuous-filament thread body, the disposition is selected from the group consisting of a row disposition, a staggered disposition, an overlapping disposition, an offset disposition, an offset and partially overlapping disposition, a zigzag disposition, a random or arbitrary disposition, a meandering disposition, a serpentine disposition, a sinus disposition, a spiral disposition, a helical disposition, and combinations thereof.

4. The continuous-filament thread according to claim 1, wherein the barbs are arranged in a unidirectional disposition or in a multidirectional or bidirectional disposition on the continuous-filament thread body and the continuous-filament thread body comprises repeating units of unidirectionally, multidirectionally or bidirectionally arranged barbs.

5. The continuous-filament thread according to claim 1, wherein the continuous-filament thread body comprises sections that are free of barbs.

6. The continuous-filament thread according to claim 1, wherein the continuous-filament thread body comprises sections having barbs interspaced by sections having no barbs.

7. The continuous-filament thread according to claim 1, wherein the continuous-filament thread body is a mass thread body.

8. The continuous-filament thread according to claim 1, wherein the continuous-filament thread body is a hollow, tubular continuous-filament thread body.

9. A barbed suture comprising at least one elongate suture body comprising a plurality of barbs which project from the at least one suture body and are formed from the continuous-filament thread according to claim 1.

10. The barbed suture according to claim 9, wherein the barbs are arranged in a unidirectional, multidirectional or bidirectional disposition on the at least one suture body.

11. The barbed suture according to claim 9, wherein the at least one suture body comprises sections that are free of barbs.

12. The barbed suture according to claim 9, wherein the suture comprises sections having barbs interspaced by sections having no barbs.

13. The barbed suture according to claim 9, wherein the suture comprises at least one chemical, physical or mechanical junction selected from the group consisting of a knot, a knob, a welded junction, an ultrasonic welded junction, a soldered junction, a bonded junction, a joining flange, a T-fitting, and combinations thereof.

14. The barbed suture according to claim 9, wherein the suture comprises a junction in the center of the suture.

15. The barbed suture according to claim 9, wherein the suture comprises a plurality of suture bodies joined by a common junction.

16. A surgical instrument comprising a surgical needle and a surgical suture combination comprising a barbed suture according to claim 9.
